(19) United States
(12) Patent Application Publication
(10) Pub. No.: US 2024/0148526 A1
(43) Pub. Date: May 9, 2024

(54) Helical Wrap For Stent Wire Attachment

(71) Applicant: MicroVention, Inc., Aliso Viejo, CA (US)

(72) Inventor: Ponaka Pung, Signal Hill, CA (US)

(73) Assignee: MicroVention, Inc., Aliso Viejo, CA (US)

(21) Appl. No.: 18/503,891

(22) Filed: Nov. 7, 2023

Related U.S. Application Data
(60) Provisional application No. 63/382,661, filed on Nov. 7, 2022.

(51) Int. Cl. A61F 2/90; A61L 31/02

(52) U.S. Cl. CPC A61F 2/90; A61L 31/022; A61F 2220/005

(57) ABSTRACT

A vascular implant may comprise at least a first layer having one or more drawn fill tube wires (DFT wires) and at least a second layer having one or more non-DFT wires. The first layer may be braided from only a single DFT wire, and the second layer may be braided from a plurality of non-DFT wire. A vascular implant may also include a connecting wire composed of a shape memory alloy and that is shape set prior to connection to one or more implant layers composed of DFT wires.

### RELATED APPLICATIONS

This application claims benefit of and priority to U.S. Provisional Application Ser. No. 63/382,661 filed Nov. 7, 2022 entitled Helical Wrap for Stent Wire Attachment, which is hereby incorporated herein by reference in its entirety.

### BACKGROUND

Vascular prostheses such as stents and stent-grafts are used for a variety of reasons in the vasculature. A non-exhaustive list includes propping open diseased or occluded vessels to promote blood flow, flow diversion involving diverting flow away from target areas such as aneurysms, and retaining material (e.g., embolic material) within a treatment site to promote localized occlusion within a region.

Visualization remains important for the delivery of vascular prostheses so that a surgeon can confirm proper placement of the device in the vasculature. Typically, radiography is used for such visualization, which is an imaging technique using X-rays, gamma rays, or similar ionizing radiation and non-ionizing radiation to view the form of an object within a patient. Specific types of radiography include static X-rays, CT scans, and fluoroscopy. For structures to be displayed by most radiography, it must be relatively radiopaque. For that reason, one or more radiopaque components are often included on most implantable vascular prostheses.

### SUMMARY

The disclosure generally relates to a vascular prosthesis composed of one or more drawn filled tube wires (DFT wires).

One aspect of the disclosure is generally directed to an implant having at least one braided layer composed of one or more DFT wires and at least one braided inner layer composed of one or more wires (e.g., DFT wires or non-DFT wires).

The implant may have a variety of different layer configurations of braided DFT wire layers and non-DFT wire layers. For example, the implant may have two braided layers in which the outer layer is composed of DFT wires and the inner layer is composed of non-DFT wires, or in which the outer layer is composed of non-DFT wires and the inner layer is composed of DFT wires. In another example, the implant may have three braided layers in which the layer composed of DFT wires is the outer layer, middle layer, or inner layer, and the two remaining layers composed of non-DFT wires are the remaining layers. In another example, the implant may have three braided layers in which a layer composed of non-DFT wires is the outer layer, the middle layer, or inner layer, and the two remaining layers composed of DFT wires are the remaining layers. In yet another example, the implant may be composed of four or more layers with layers alternating between braided DFT wire layers and non-DFT wire layers (e.g., the DFT wire layer may comprise the outermost layer or the non-DFT wire layer may compose the non-DFT wire layer).

In addition to having different combinations of DFT wire and non-DFT wire layers, the implant layers may have different lengths relative to each other. For example, a braided DFT wire layer may extend beyond the proximal and/or distal end of the non-DFT wire layer(s) or a non-DFT wire layer may extend beyond the proximal and/or distal end of the DFT wire layer(s).

Another aspect of the disclosure is generally directed to a vascular implant (e.g., a stent or graft) having one or more connecting wires that are non-DFT wires, preferably composed of a shape memory material, and that may be pre-shaped (e.g., heat set) to a desired secondary shape and then connected and/or braided into one or more braided layers of the implant. By pre-shaping the connecting wire to a desired secondary shape, the connecting wire may provide additional force to an implant to achieve its desired expanded configuration size and may help maintain the expanded configuration, particularly in tortuous vessels. As discussed herein, DFT wires may be relatively flexible as compared to non-DFT wires, especially after being heat set. Hence, a connecting wire that is pre-shaped to a desired expanded size may help force the other layers of the implant, including those with DFT wires, to achieve or maintain a desired radial size and potentially better anchor within a patient's vasculature.

The implant may include a single layer, two layers, three layers, or more than three layers. The implant may also include at least one layer braided from DFT wire, and optionally a plurality of layers (e.g., 2 or 3) that are braided from DFT wire. As in previously discussed examples, the remaining layers may be composed of non-DFT wires.

The pre-shaped connecting wire may form a helical shape or may be one or more circular shapes. A single connecting wire may be used with an implant or a plurality of connecting wires may be used with an implant. The one or plurality of connecting wires may each extend along the entire length of the stent (or most of the length of the stent) or the one or plurality of connecting wires may extend along only a fraction of the length of the implant (e.g., a quarter, third, half, or three-quarters of the length of the implant).

A plurality of separate connecting wires may be used in a non-overlapping configuration. For example, one connecting wire may extend along a first half of an implant and a second connecting wire may extend along a second half of an implant. Similar configurations may be possible for 3, 4, 5, 6, or more connecting wires. Alternatively, a plurality of connecting wires can be arranged so that only portions of each connecting wire overlap in their position along the implant length.

The connecting wires may be connected to one or a plurality of implant layers by interweaving the one or more connecting wires through each of the implant layers and/or by connecting the one or more connecting wires via a connection mechanism to wire locations on the stent, such as welding, rings, wire coils, wire ties, coiling the ends of the connecting wires, or similar techniques. The connecting wires may be used in only a single layer stent example to help open the implant, a two-layer stent example to help connect the layers, or a three or more layer stent example to help connect at least two of the implant layers generate additional radial opening force.

In one example, the connecting wire is any shape memory alloy, such as Nitinol. The connecting wire may be pre-shaped by winding on a mandrel to form a desired size and pattern, and then heat set to establish the desired secondary shape of the connecting wire. The connecting wire may then be connected (e.g., interwoven or fixed to) the one or plurality of layers of an implant. The connecting wire may have a similar shape as one or more portions of the wire of a layer of an implant (i.e., it may closely follow the shape of a portion of one of the implant's wires) or it may have a different pattern/shape than the wire portions of an implant.

The present disclosure includes connection mechanisms that include any combination of adhesive, coils, laser welding, beads, sleeves, tubes, or similar structures. For example, a first wire of a first material and a second wire of a second material may be connected or bound together via one or more of the following: a third wire or wires coiled around both the first and second wires, one or a plurality of sleeves positioned around the first and second wires, and/or one or a plurality of tubes positioned around the first and second wires. Adhesive may optionally be applied to the first and second wires at a region that the third wire(s), sleeve(s), or tube(s) are located. A stop member may optionally be included with any of the prior examples where the stop member is located on only the first wire adjacent to the third wire(s), sleeve(s), or tube(s), on only the second wire adjacent to the third wire(s), sleeve(s), or tube(s), or on both the first wire and the second wire adjacent to ends of the third wire(s), sleeve(s), or tube(s). Each of the stop members may include one or more wire coils, one or more laser weld locations, one or more beads, one or more sleeves, one or more tubes, or similar shapes. In any of the prior examples, the first material may be a shape memory alloy, such as Nitinol, and the second material may be any of the DFT wire examples of this disclosure. In any of the prior examples, the first wire may be about parallel with the second wire, both extending in generally the same orientation and direction.

In some aspects, the techniques described herein relate to a stent including: a first wire; a second wire; and, a wire connection structure fixed at an end region of the second wire and fixed to the first wire.

In some aspects, the techniques described herein relate to a stent, wherein the first wire is about parallel to the second wire.

In some aspects, the techniques described herein relate to a stent, wherein the first wire is braided to form a first stent layer and wherein the second wire is a connecting wire interwoven with the first wire.

In some aspects, the techniques described herein relate to a stent, wherein the connecting wire is interwoven with the first stent layer and with a second stent layer.

In some aspects, the techniques described herein relate to a stent, wherein the first stent wire includes a DFT wire and wherein the second stent wire includes a Nitinol wire.

In some aspects, the techniques described herein relate to a stent, wherein the wire connection structure includes a primary wire coil wrapped around the first wire and the second wire, near the end region of the second wire.

In some aspects, the techniques described herein relate to a stent, wherein the coil includes 1, 2, 3, 4, 5, 6, 7, 8, 9, or 10 loops.

In some aspects, the techniques described herein relate to a stent, wherein the primary wire coil is composed of a tantalum wire.

In some aspects, the techniques described herein relate to a stent, further including adhesive located underneath the primary wire coil.

In some aspects, the techniques described herein relate to a stent, further including one or a plurality of stop members connected to the first wire, the second wire, or separately to the first wire around the second wire; wherein the one or plurality of stop members are adjacent to the primary wire coil.

In some aspects, the techniques described herein relate to a stent, wherein the first wire includes a first stop member adjacent a first end of the primary wire coil and a second stop member adjacent a second end of the primary wire coil; and wherein the second wire includes a third stop member adjacent the first end of the primary wire coil and a fourth stop member adjacent the second end of the primary wire coil.

In some aspects, the techniques described herein relate to a stent, wherein the one or more secondary stop members include one or a plurality of secondary coils, one or a plurality of sleeves, one or a plurality of tubes, one or a plurality of beads, one or a plurality of welds, or one or a plurality of attached enlargements.

In some aspects, the techniques described herein relate to a stent, wherein the wire connection structure includes a first weld that is welded to the first wire and the second wire, and a second weld wired to the first wire and the second wire at the end region of the second wire.

In some aspects, the techniques described herein relate to a stent, wherein the first weld is spaced apart from the second weld; and wherein adhesive is located on the first wire and the second wire between the first weld and the second weld.

In some aspects, the techniques described herein relate to a stent, wherein the connecting wire is connected to an end loop of the first stent layer formed from the first wire.

In some aspects, the techniques described herein relate to a stent, wherein the wire connection structure further includes a first wire connection structure connecting a first end region of the connecting wire to a first end loop of the stent, and a second wire connection structure connecting a second end region of the connecting wire to a second end loop of the stent.

In some aspects, the techniques described herein relate to a stent including: a first wire including a first material; a second wire that is about parallel to the first and including a second material; and, a wire connection structure fixed at an end region of the second wire and fixed to the first wire; the wire connection structure including a primary wire coil and one or more stop members located longitudinally adjacent the primary wire coil.

In some aspects, the techniques described herein relate to a stent, wherein the stop members include one or a plurality of secondary coils, one or a plurality of sleeves, one or a plurality of tubes, one or a plurality of beads, one or a plurality of welds, or one or a plurality of attached enlargements.

In some aspects, the techniques described herein relate to a stent, further including adhesive located at least underneath the primary wire coil.

In some aspects, the techniques described herein relate to a stent including: a first wire; a second wire; and, a wire connection structure means for fixing an end region of the second wire to the first wire.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. Reference is being made to the accompanying drawings, in which:

FIG. 1 illustrates a cross section of a DFT wire used in a DFT stent, according to one example.

FIG. 2A illustrates a side view of a dual layer DFT stent, according to one example.

FIG. 2B illustrates a photograph side view of the dual layer DFT stent of FIG. 2A, according to one example.

FIG. 3A illustrates an end view of the dual layer DFT stent of FIG. 2A, according to one example.

FIG. 3B illustrates a photograph end view of the dual layer DFT stent of FIG. 2A, according to one example.

FIG. 3C illustrates a magnified view of end loops of a dual layer DFT stent, according to one example.

FIG. 4 illustrates a magnified view of the dual layer DFT stent of FIG. 2A, according to one example.

FIG. 5 illustrates a magnified view of the dual layer DFT stent of FIG. 2A, according to one example.

FIG. 6 illustrates a magnified view of the dual layer DFT stent of FIG. 2A, according to one example.

FIG. 7 illustrates a magnified view of the dual layer DFT stent of FIG. 2A, according to one example.

FIG. 8 illustrates an end view of another example of a stent, according to one example.

FIG. 9 illustrates an end view of another example of a stent, according to one example.

FIG. 10 illustrates a connecting wire on a mandrel, according to one example.

FIG. 11 illustrates a connecting wire connected to a stent wire, according to one example.

FIG. 12 illustrates a side view of a single layer stent, according to one example.

FIG. 13 illustrates an end view of a DFT stent end loop configuration, according to one example.

FIG. 14 illustrates an end view of a DFT stent end loop configuration, according to one example.

FIG. 15 illustrates a planar view of a DFT stent end loop configuration, according to one example.

FIG. 16 illustrates a side view of a stent with a reinforcing member, according to one example.

FIG. 17 illustrates an enlarged view of a stent with a reinforcing member, according to one example.

FIG. 18 illustrates a side view of a connection structure for wires of a stent according to one example.

FIG. 19 illustrates a side view of a connection structure for wires of a stent according to one example.

FIG. 20 illustrates a side view of a connection structure for wires of a stent according to one example.

FIG. 21 illustrates a side view of a connection structure for wires of a stent according to one example.

FIG. 22 illustrates a side view of a connection structure for wires of a stent according to one example.

FIG. 23 illustrates a side view of a connection structure for wires of a stent according to one example.

FIG. 24 illustrates a side view of a connection structure for wires of a stent according to one example.

FIG. 25A illustrates a side view of stent with connection structures according to one example.

FIG. 25B illustrates a magnified view of a connection structure of FIG. 25A.

### DETAILED DESCRIPTION

It will be appreciated by persons skilled in the art that the present disclosure is not limited to what has been particularly shown and described herein. A variety of modifications and variations are possible in view of the above teachings without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value.

The present specification also describes several different features of stents, such as different wire materials of different layers, different layer arrangements, different layer lengths, different connections between layers, and other features. Any of these aspects can be used and interchanged with each other. Hence, while every permutation of features is not specifically described, such combinations are specifically contemplated as being part of the disclosure and supported by the specification.

This specification also refers to the use of drawn filled tube wires (DFT wires) and non-DFT wires. The non-DFT wires 16 can be composed of any material typically used for medical devices, including shape memory alloys (e.g., Nitinol), stainless steel, cobalt-chromium, polymers, or other materials. Shape memory alloys, and especially Nitinol, may be preferable in some examples. These non-DFT wires 16 are generally composed of a single material through its cross section, though coatings and similar features are also possible.

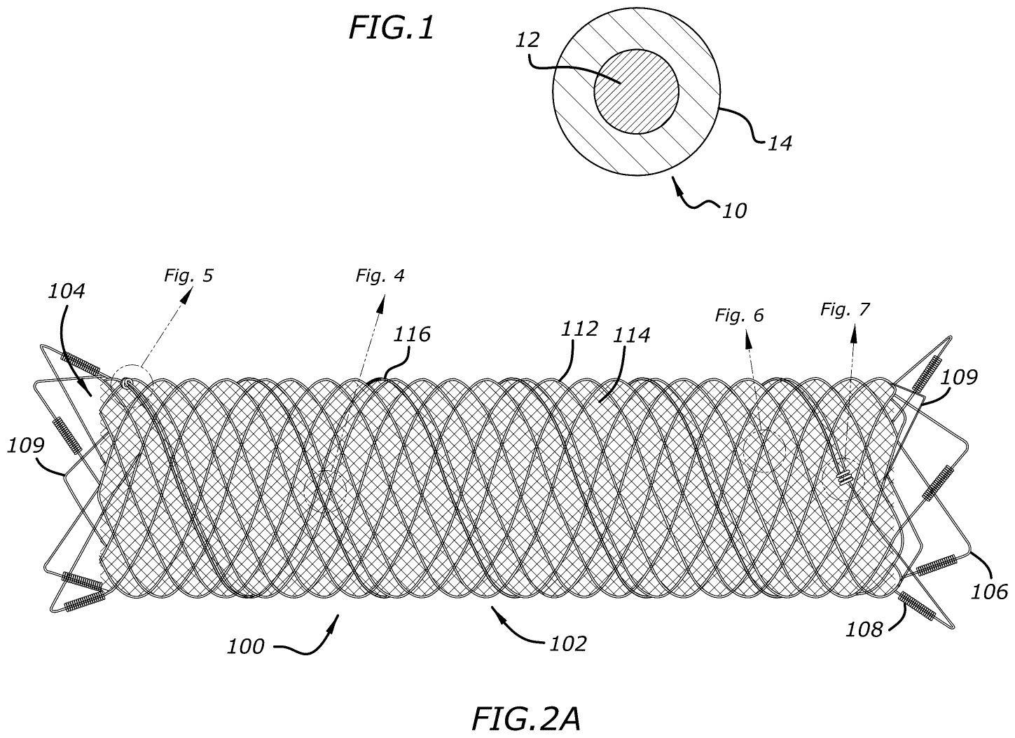

The DFT wires 10 can be composed of a variety of different materials with different cross-sectional thicknesses. For example, FIG. 1 illustrates a cross section of a DFT wire 10 having an inner core 12 composed of a first material and an outer jacket 14 composed of a second material. In another example, the outer jacket 14 may alternatively be composed of multiple layers of different material (e.g., two or more layers over inner core 12). Either of the inner core 12 and the outer jacket 14 can be composed of radiopaque materials (such as platinum, gold, tantalum, palladium, or similar known radiopaque materials). Either of the inner core 12 and the outer jacket 14 can be composed of non-radiopaque materials (i.e., materials with a relatively low or no radiopaque properties). Such non-radiopaque materials may include, e.g., stainless steel, cobalt-chromium, or shape memory alloys such as Nitinol. In one example, the inner core 12 may be composed of radiopaque material(s) and the outer jacket 14 may be composed of non-radiopaque materials. In another example, the inner core 12 may be composed of non-radiopaque materials and the outer jacket 14 may be composed of non-radiopaque materials.

In one example, the inner core 12 may be composed of a radiopaque material and the outer jacket 14 may be composed of a shape memory alloy such as Nitinol. The radiopaque material promotes visualization of the DFT wire 10, while the outer jacket 14 allows for good pliability and the ability to have a memorized shape (e.g., via being heat-set). In another example, inner core 12 may be composed of platinum or tantalum, while the outer jacket 14 may be composed of Nitinol-1 or Nitinol-2.

The inner core 12 may have a cross sectional shape that is circular, elliptical, or ovular, though a variety of other shapes can be used, such as rectangular, triangular, or the like. The outer jacket 14 may be tubular in shape with an inner diameter that closely matches an outer diameter of the inner core 12. Put differently, the outer jacket 14 may include an internal lumen through which the inner core 12 extends.

Additionally, DFT wires 10 may sometimes exhibit a higher degree of bendability and reduced stiffness than a purely metallic shape memory wire once heat treatment/heat-setting occurs. This may be generally unexpected since inclusion of a radiopaque material in the inner core 12 (depending on which particular material is used) can generally be stiffer in comparison to the metallic shape memory outer jacket 14. However, the inclusion of two separate materials in creating a single wire can alter the material characteristics of the combined wire shape. Due to these characteristics, when DFT wires 10 are used in a stent, design aspects of the stent may need to compensate for this increased flexibility, especially to promote proper deployment and proper apposition of the DFT stent at the treatment site to prevent stent migration. The examples presented herein address these and other issues to create a usable DFT stent.

The outer diameter of the DFT wire 10 may have a wide range of diameters, depending on its use within a stent. For example, the DFT wire 10 may have a diameter within an inclusive range of about 0.001 inch to 0.004 inch, or about 0.0025 inch to about 0.003 inch. The inner core 12 and outer jacket 14 of the DFT wire 10 may be composed of different percentages of the cross section of the DFT wire 10 based on cross-sectional width or diameter. For example, the inner core 12 may be within an inclusive range of 5% to 30% of the cross-sectional width or diameter of the DFT wire 10 with the remaining percentage being the outer jacket 14 (i.e., 95% to 70%). In a more specific example, the ration may be 10% inner core 12 cross sectional width or diameter and 90% outer jacket 14 cross sectional width or diameter.

In some examples, the total cross-sectional width or diameter of the DFT wire 10 is within an inclusive range of about 0.0018 inch to about 0.0022 inch. In some examples, the inner core 12 (e.g., composed of a radiopaque material) has a width or diameter within an inclusive range of about 0.0005 inch to about 0.001 inch, or an inclusive range of about 0.0008 inch to about 0.0009 inch.

Any of the wires 10, 16 used in a stent may be functionalized, for example with poly (MEA-co-APMA).

One aspect of the present disclosure is generally directed to a stent having at least one braided layer composed of one or more drawn filled tube wires (DFT wires) and at least one braided inner layer composed of one or more wires (e.g., DFT wires or non-DFT wires).

The stent may have a variety of different layer configurations of braided DFT wire layers and non-DFT wire layers. For example, the stent may have two braided layers in which the outer layer is composed of one or more DFT wires, and the inner layer is composed of non-DFT wires, or that the outer layer is composed of non-DFT wires and the inner layer is composed of DFT wires. In another example, the stent may have three braided layers in which the layer composed of DFT wires is the outer layer, middle layer, or inner layer, and the two remaining layers composed of non-DFT wires are the remaining layers. In another example, the stent may have three braided layers in which a layer composed of non-DFT wires is the outer layer, the middle layer, or inner layer, and the two remaining layers composed of DFT wires are the remaining layers. In yet another example, the stent may be composed of four or more layers with layers alternating between braided DFT wire layers and non-DFT wire layers (e.g., the DFT wire layer may composed the outermost layer, or the non-DFT wire layer may compose the non-DFT wire layer).

In addition to having different combinations of DFT wire and non-DFT wire layers, the stent layers may have different lengths relative to each other. For example, a braided DFT wire layer may extend beyond the proximal and/or distal end of the non-DFT wire layer(s) or a non-DFT wire layer may extend beyond the proximal and/or distal end of the DFT wire layer(s).

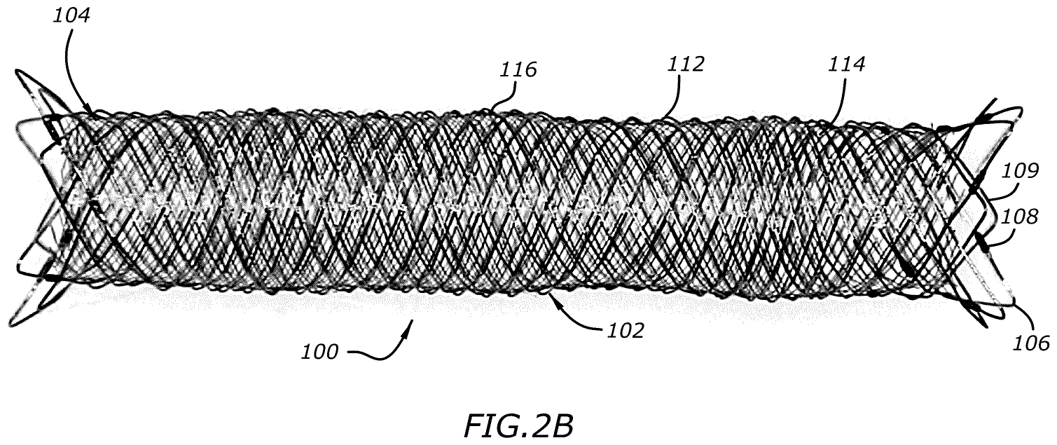

FIGS. 2A-7 illustrate one specific example of a stent 100 having at least one layer composed of one or more DFT wires 10 and at least one layer composed of one or more non-DFT wires 16. More specifically, the stent 100 may include a braided outer layer 102 forming a tubular shape composed of one or more outer wires 112 that are DFT wires 10 and a braided inner layer 104 forming a tubular shape within the outer layer 102 that is composed of one or more inner wires 114 that are non-DFT wires 16. The DFT wires 10 may have any of the previously discussed characteristics, but preferably have an inner core 12 composed of a radiopaque material and an outer jacket 14 composed of a shape memory alloy (e.g., Nitinol).

The use of DFT wires 10, particularly with radiopaque materials comprising the inner core 12, can provide several advantages. First, the DFT wires 10 of the outer layer 102 may be radiopaque and therefore show up on radiography visualization. Unlike the use of relatively small radiopaque markers, the entire outer layer 102 may be visualized which may allow for a physician to better view and place the stent 100. Since radiopaque markers may not be necessary, the lack of such markers may further decrease the profile or thickness of a stent.

Additionally, when a radiopaque material is used in the DFT wire(s) 10, it may have a relatively higher flexibility or bendability than many non-DFT wires 16 composed of shape memory alloys (e.g., Nitinol) because of the properties of the material used in the DFT wire 10 and/or after being heat set to impart a shape to the wire. Hence, a stent layer composed of one or more DFT wires 10 may sometimes better conform to a shape of a tortuous anatomical site within a patient.

The stent 100 may also include several other features discussed further below that may be helpful in connection with DFT wire 10 and non-DFT wire 16 stent layers, though they are not necessarily required. Note, Areas labeled FIG. 4-7 in FIG. 2A correspond to magnified views in FIGS. 4-7, respectively, and FIG. 2B illustrates a photograph view of FIG. 2A.

In one example, the stent 100 may include a tubular shaped outer layer 102 and a tubular shaped inner layer 104 that is attached to the outer layer 102. The outer layer 102 can be configured to anchor the stent 100 within a patient while the inner layer 104 may be less porous than the outer layer 102 so as to help divert or prevent blood flow from passing through.

Both the inner layer 104 and the outer layer 102 may be braided in a helical braiding pattern so that the wires have the same or similar braid angles. This may allow both layers 102 and 104 to increase and decrease in length at the same or similar rate when the stent 100 radially expands or contracts between its radially compressed configuration and its radially expanded configuration. Alternatively, the layers 102, 104 may have different braid patterns and/or braid angles.

The outer layer 102 may have a larger pore size or a lower pick per inch (PPI) than the inner layer 104. In one example, the pores may be sized within an inclusive range of about 0.3 mm to about 0.5 mm when the stent 100 is in its expanded configuration. In another example, the braided tubular portion of the outer layer 102 may have a pick per inch within an inclusive range of about 60 PPI to about 85 PPI, and more specifically about 72. However, in some examples, the pore/cell sizes and/or the pick per inch of the respective layers 102, 104 may be the same or similar.

The outer wire 112 of the outer layer 102 may have a larger diameter than the inner wire 114 of the inner layer 104. For example, the outer wire 112 of the outer layer 102 may have a diameter within an inclusive range of about 0.001 inch to 0.004 inch, or about 0.0025 inch to about 0.003 inch. In one example, the outer wire 112 of the outer layer 102 may have a diameter of about 0.0016 inch throughout its braided tubular portion and a diameter of about 0.0020 inch along portions of the outer wire 112 forming its end loops 106, 109.

The outer layer 102 may be braided from a single outer wire 112 (e.g., a DFT wire 10) into its tubular shape. Alternatively, the outer layer 102 may be braided from a plurality of outer wires 112 (e.g., DFT wires 10) into its tubular shape. Again, in other examples, these outer layer configurations may use non-DFT wire 16 instead. Example diameter sizes for the outer layer 102 in its expanded configuration include 2.5 mm-3.0 mm, 3.5 mm-4.5 mm, 4.5 mm-5.0 mm, 5.0 mm-5.5 mm, 5.5 mm-6.0 mm, and 6.0 mm-8.0 mm with various lengths.

The inner layer 104 may be braided from a single inner wire 114 (e.g., a non-DFT wire 16) into its tubular shape. Alternatively, the inner layer 104 may be braided from a plurality of inner wires 114 (e.g., non-DFT wires 16) into its tubular shape. Again, in other examples, these inner layer configurations may use DFT wire 10 instead. The inner layer 104 may form a braided, tubular shape that may be sized to expand to an outer diameter equal to or almost equal to the inner diameter of the outer layer 102. The inner layer 104 can be composed of one or more inner wires 114 (e.g., 20, 24, 36 wires) braided with each other to form its tubular shape. In either wire example, the wire diameter may be about 0.00085 inch and be braided to form about 165 picks per inch in an example.

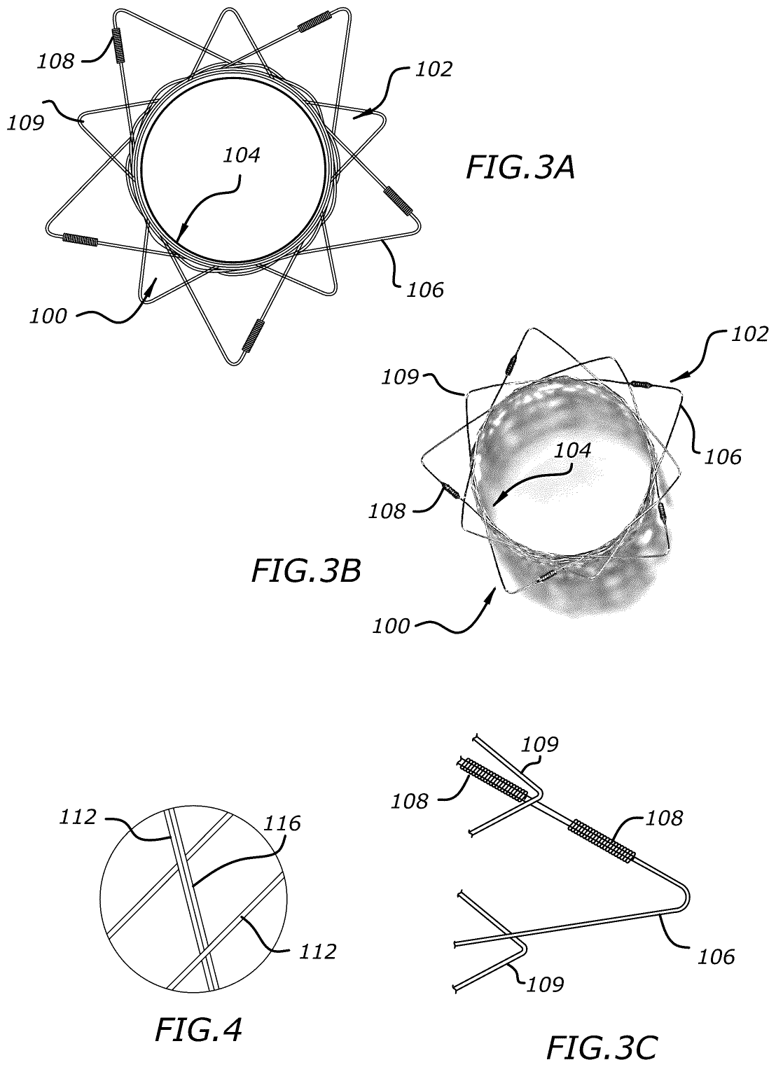

The outer layer 102 may form a braided, tubular shape with a plurality of end loops that can be the same size or different sizes. The loops can be located on the proximal end, the distal end, or both ends. Each end of the braided tubular portion may have, for example, 3, 4, 5, 6, 7, 8, 9, 10, 11, 12, or more loops. The loops may have larger and smaller sizes, such as larger loops 106 and smaller loops 109 in FIG. 3A. These larger and smaller loops 106, 109 may form an alternating pattern as seen in FIG. 3A, where five larger and five smaller loops are interposed with each other. Note, FIG. 3B illustrates a photograph view of FIG. 3A.

As seen in FIG. 6, the ends of the outer wires 112 may be located near each other, such as about three-quarters along the stent's 100 length. The wire ends may be positioned to overlap each other and then one or more (e.g., four) laser welds 112A may be created to connect the portions of the wire 112, thereby preventing the ends or edges of the wire 112 from being easily exposed in a location that may cause damage to the patient. Alternatively, other connection mechanisms are possible for these wire ends, such as being tied together or positioned under a separate coil or band.

When DFT wires 10 are used that include a radiopaque inner core 12, additional radiopaque markers may not be necessary. However, depending on which layers incorporate the radiopaque DFT wires 10, radiopaque markers may be helpful, particularly on the ends of the stent 100 to help identify where the stent 100 terminates. In one example such as shown in FIG. 3C, the end loops 106 of the outer layer 102 may also include one or more wire coils 108 (alternatively sleeves, tubes, or similar shapes) wrapped around a portion of the outer wire 112 of an end loop 106. The wire coils 108 may be composed of a radiopaque material such as tantalum to help indicate the ends of the stent 100 under imaging and provide additional anchoring force. However, non-radiopaque material may alternatively be used. In one example, each end may include four tantalum wire coils 108, the coils 108 may be each positioned near the apex or near the furthermost end of each loop 106, and/or each coil 108 may be formed from tantalum wire having a diameter of about 0.0015 inch. Alternatively, the wire coils 108 can be composed of a non-radiopaque material such as Nitinol and may be provided only for anchoring purposes.

The coils 108 can be placed at any location on the loops 106 (or optionally the loops 109). As seen in FIG. 3C, the coil 108 can be positioned closer to the terminal end of a loop or closer to the body of the stent. The example of FIGS. 2A-7 illustrate only the larger loops 106 having coils 108 positioned relatively closer to the terminal ends of the loops 106. In another example, the loops 106 may be positioned closer to the body of the stent (i.e., the left-most coil 108 as shown in FIG. 3C). In this example, the coil 108 is further positioned within the smaller loop 109 such that it remains within the smaller loop 109 when the stent is both in its radially compressed configuration and radially expanded configuration. Since the loops 106 and 109 may move somewhat relative to each other during radial expansion, this positioning may help prevent the coil 108 from moving against the wires of the smaller loop 109, allowing for a smoother opening movement of the stent 100. In another example, a loop may include two coils 108 at both inner and outer locations, as seen in FIG. 3C.

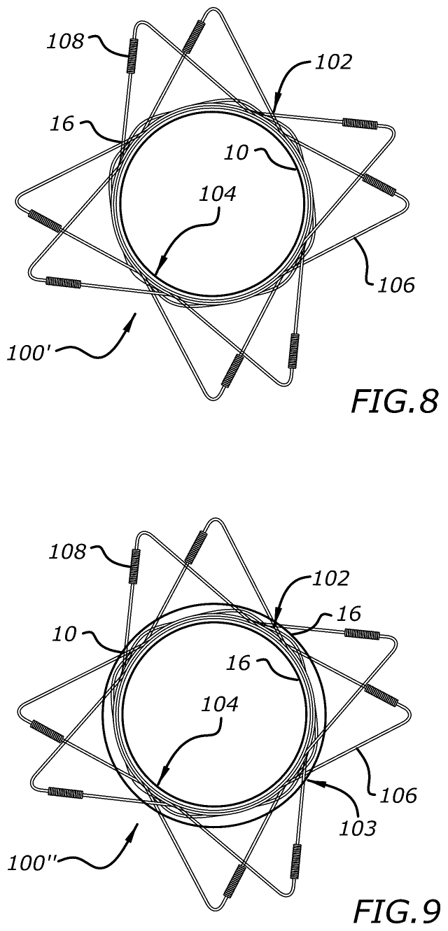

FIG. 8 illustrates another example of a stent 100′ that is generally similar to the previously described stent 100 example but in which the outer layer 102 may be composed of non-DFT wires 16 and the inner layer 104 is composed of DFT wires 10.

FIG. 9 illustrates another example of stent 100″ that is generally similar to the previously described stent 100 example, but which includes a third outer layer 103. Both the third outer layer 103 and the inner layer 104 may be composed non-DFT wires 16 and the middle layer 102 may be composed of DFT wires 10. Alternatively, any combination of DFT wires 10 and optionally non-DFT wires 16 can be used for each layer. For example, all layers may be composed of DFT wires 10, only one of the layers 102, 103, 104 can be composed of DFT wires 10 with the remaining being non-DFT wires 16, or two of the layers 102, 103, 104 can be composed of DFT wires 10 with the remaining layer being non-DFT wires 16.

Another aspect of the disclosure is generally directed to a vascular device (e.g., a stent or graft) having one or more connecting wires 116 that may be non-DFT wires 16, preferably composed of a shape memory material, and that may be pre-shaped (e.g., heat set) to a desired secondary shape and then connected and/or braided into to one or more braided layers of a stent. By pre-shaping the connecting wire 116 to a desired secondary shape, the connecting wire 116 may provide additional force to a stent to achieve and/or maintain its desired open configuration size. As previously discussed, DFT wires 10 may be relatively flexible as compared to non-DFT wires 16, depending on their material composition. Hence, a connecting wire 116 that is pre-shaped to a desired expanded size may help force the other layers of the stent, including those with DFT wires 10 to achieve a desired radial size and potentially better anchor within a patient's vasculature.

The stent may include a single layer, two layers, three layers, or more than three layers. The stent may also include at least one layer braided from DFT wire 10, and optionally a plurality of layers (e.g., 2 or 3) that are braided from DFT wire 10. As in previously discussed examples, the remaining layers may be composed of non-DFT wires 16.

The pre-shaped connecting wire 116 may form a helical shape or may be one or more circular shapes. A single connecting wire 116 may be used with a stent or a plurality of connecting wires 116 may be used with a stent. The one or plurality of connecting wires 116 may each extend along the entire length of the stent (or most of the length of the stent) or the one or plurality of connecting wires 116 may extend along only a fraction of the length of the stent (e.g., a quarter, third, half, or three-quarters of the length of the stent).

A plurality of separate connecting wires 116 may be used in a non-overlapping configuration. For example, one connecting wire 116 may extend along a first half of a stent and a second connecting wire 116 may extend along a second half of a stent. Similar configurations may be possible for 3, 4, 5, 6, or more connecting wires 116. Alternatively, a plurality of connecting wires 116 can be arranged so that only portions of each connecting wire overlap in their position along the stent length.

The connecting wires 116 may be connected to one or a plurality of stent layers by interweaving the one or more connecting wires 116 through each of the stent layers and/or by connecting the one or more connecting wires 116 via a connection mechanism to wire locations on the stent, such as welding, rings, wire coils, wire ties, coiling the ends of the connecting wires 116, or similar techniques. The connecting wires 116 may be used in only a single layer stent example to help open the stent, a two-layer stent example to help connect the layers, or a three or more layer stent example to help connect at least two of the stent layers generate additional radial opening force.

In one example, the connecting wire 116 may be any shape memory material, such as Nitinol. The connecting wire 116 may be pre-shaped by winding on a mandrel to form a desired size, shape, and pattern, and then heat set to establish the desired secondary shape of the connecting wire 116. The connecting wire 116 may then be connected (e.g., interwoven or fixed to) the one or plurality of layers of a stent. The connecting wire 116 may have a similar shape as one or more portions of the wire of a layer of a stent (i.e., it may closely follow the shape of a portion of one of the stent's wires) or it may have a different pattern/shape than the wire portions of a stent.

While the connecting wire 116 is described as being pre-shaped, it may alternatively be woven with one or more layers of a stent and heat set with the other layers of the stent.

Returning to the example of FIGS. 2A-7, the use of one or more connecting wires 116 is illustrated. In the final form of the stent 100, the one or plurality of connecting wires 116 may be positioned adjacent to a portion of outer wire 112 in a helical pattern such that is has a similar braid axis and braid angle as outer wire 112. Again, a single connecting wire 116 can be used or a plurality of connecting wires 116 in different arrangements/positions can be used.

The connecting wire 116 may be interwoven with both wires 112 and 114 of both layers 102 and 104 (e.g., an over-under pattern through both), so that both layers are relatively closely positioned adjacent to each other. The similar braid angle allows the wires 112, 114, and 116 of the stent to move in relative unison to foreshorten/lengthen during radial expansion and contraction. In one specific example, two helically woven connecting wires 116 are included, though 1, 3, 4, 5, 6, or more connecting wires 116 can also be included.

By pre shaping and/or heat setting the connecting wire(s) 116, the diameter and pitch of the helical may be defined to perform similarly to the wires 112, 114 forming the inner and outer layers 102, 104. This can be clinically advantageous, since having all components radially expand and foreshorten similarly allows the stent 100 to open and conform better to tortuous anatomies, providing better wall apposition. Thus, stent opening and stability issues may be reduced or eliminated.

In the present example of stent 100, the connecting wire 116 may be comprised of one or more Nitinol helical wires (i.e., a helical, heat set, secondary shape). The use of such Nitinol helical wires or coils instead of non-shape memory wires may allow the stent 100 to open up to a greater size (e.g., to a greater OD such as more than 5 mm) since the pre-shaped and heat set configuration of the connecting wires 116 may generate additional outward radial force, depending on the pre-shaped size of the connecting wires 116 and the otherwise expanded size of the other layers of the stent 100.

The connecting wire 116 may also or alternatively be electropolished prior to use connecting the inner and outer layers 102, 104 in some examples. However, it should be appreciated that, in some examples, the connecting wire 116 may not be electropolished prior to use connecting the inner and outer layers 102, 104.

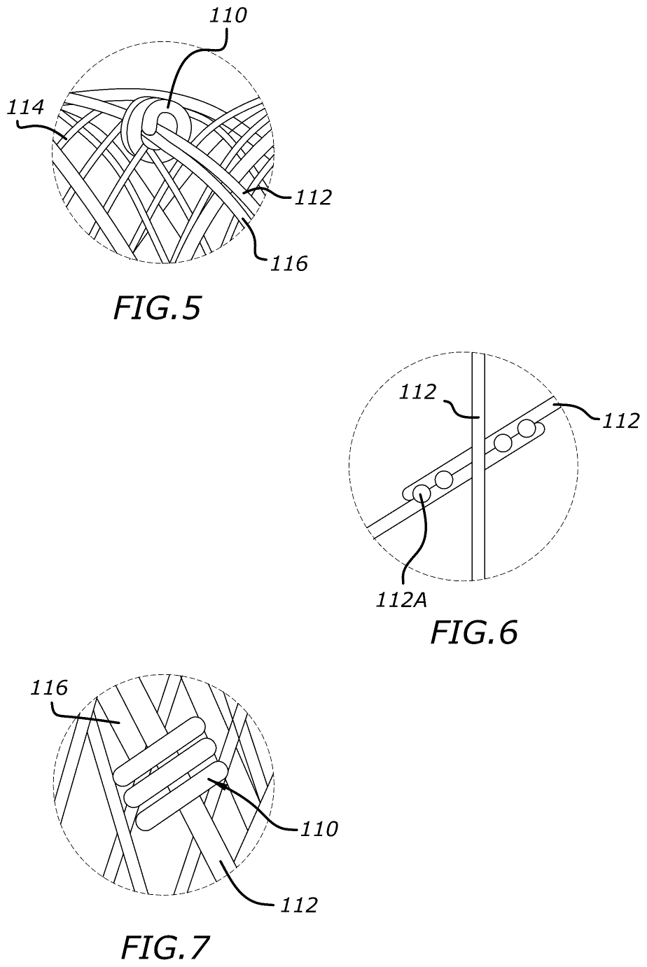

As seen in FIGS. 5 and 7, the ends of a connecting wire 116 may be connected or fixed to the outer wire 112 of the outer layer 102 (and/or optionally the inner wire 114 of the inner layer 104) to help prevent the connecting wire 116 from unwinding or coming apart from the two layers 102, 104. In one example, coiled wire ties 110 (e.g., a non-DFT wire 16 such as a tantalum or a non-super elastic alloy) may be used to connect the connecting wire 116 to the wire 112. In another example, each end of the connecting wire 116 may be wrapped around the wire 112. These coiled wire ties 110 can be connected in a manner that allows some movement of the wires 112 and 114 relative to each other or can be tightly connected in a manner that prevents movement of the wires 112 and 114 relative to each other. Coiled wire ties 110 can also optionally be used to connect both layers 102 and 104, separate of the connecting wire 116.

If a connecting wire 116 is not composed of a shape memory alloy such as Nitinol, it might instead be composed of a radiopaque material to enhance visualization. However, non-shape memory wires may be more difficult to configure to impart a desired amount of radially expansive force to achieve a relatively larger expanded stent size, particularly with one or more wires being composed of the flexible DFT wire 10. In especially tortuous or curved vessels, the two layers 102 and 104 may also exhibit force on each other and attempt to separate from each other, depending on many different factors. A pre-shape memory material/alloy such as Nitinol may act in a somewhat elastic manner when used for the connecting wire 116, returning to its original shape/configuration after the stress of deployment, whereas other materials without such super elastic properties may permanently change shape, depending on the magnitude and direction of the forces applied during delivery. In that manner, such a configuration with connecting wires composed of shape memory material may create a more resilient stent that is more resistant to damage.

Additionally, the shape memory material, such as Nitinol, of the connecting wire 116 may allow the connecting wire 116 to be heat set or pre-shaped during the manufacturing process. This pre-shaping can allow the connecting wire 116 to take on the shape of a helical coil with a predetermined diameter and pitch, similar to the wires of the inner layer 104 and outer layer 102 but, for example, with a different radial diameter so as to impart force on the other stent layers. Hence, pre-shaping the connecting wire 116 can allow for a different heat set diameter to the helical coil (or other shape) of the connecting wire 116 when in an expanded configuration versus the expanded configuration of the layers 102 and 104. In that respect, the layers 102 and 104 of the stent may be heat set after being braided, separately from the connecting wire 116, and the connecting wire 116 can be later connected and/or braided to the remaining layers 102, 104.

Hence, all three components, layers 102, 104, and the connecting wire 116, may radially expand and longitudinally contract in a similar manner, despite any sizing difference, exhibiting less resistance or force on each other. This can allow the stent 100 to open to a larger diameter (e.g., 5.0 mm or greater) than it otherwise would with connecting wire materials without super elastic properties (e.g., tantalum), and thereby provide better vessel wall apposition.

In that regard, the present example of the stent 100 may specifically include an outer layer 102 composed of a single braided DFT wire 112 with a radiopaque inner core 12 and having a first configuration (e.g., braiding/winding pattern/angle, wire diameter), an inner layer 104 composed of one or a plurality of braided inner wires 114 having a second configuration (e.g., braiding/winding pattern/angle, wire diameter), and one or a plurality of pre-shaped connecting wires 116 (e.g., Nitinol) that connect the inner and outer layers 102, 104 together (e.g., a helical woven wire and/or a coiled wire).

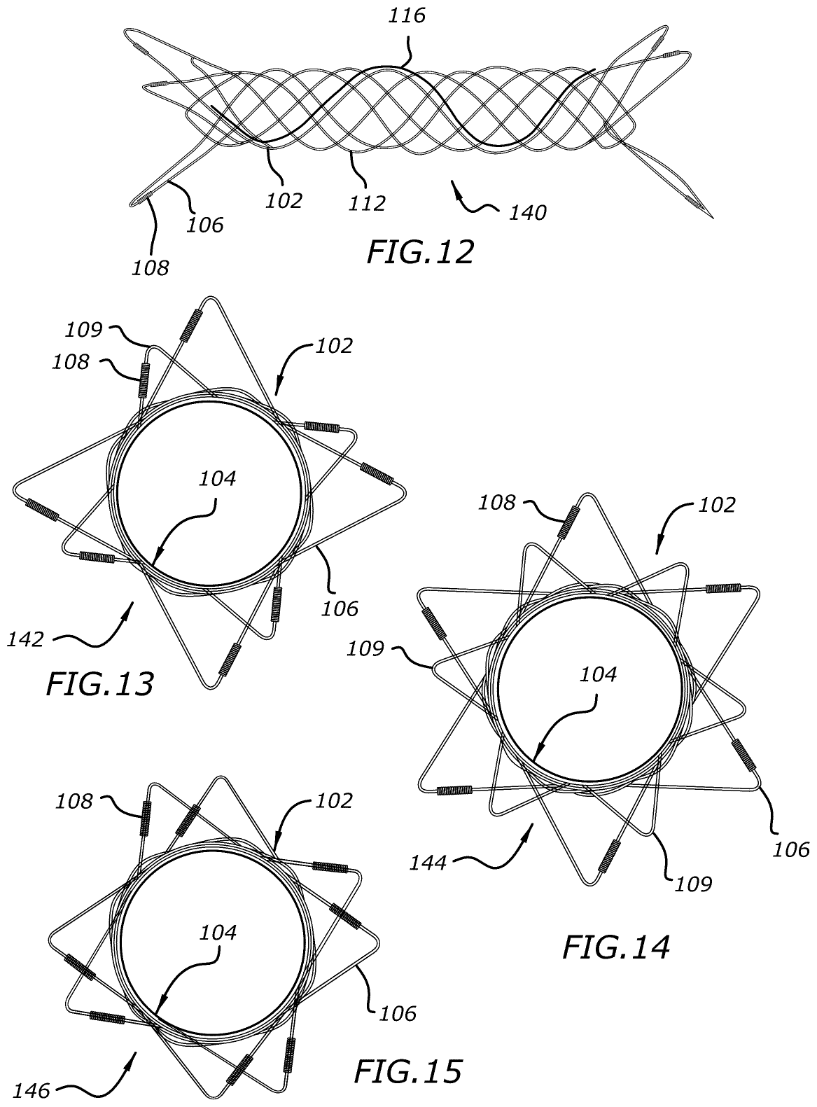

As previously discussed, the connecting wire 116 may be used with other stent examples having other layer configurations. For example, FIG. 12 illustrates a single layer stent 140 that is generally similar to the outer layer 102 of the previously discussed stent 100. The wires 112 may be heat set DFT wires 10 and therefore may have a relatively higher flexibility. One or a plurality of connecting wires 116 may be connected to and/or interwoven with the stent 140 in any of the previously discussed arrangements to provide the previously discussed performance advantages (e.g., expansion and anchoring).

In other examples, the stents 100′ and 100″ of FIGS. 8 and 9 may also include one or a plurality of connecting wires 116 similar to any of the previously discussed arrangements. The connecting wires 116 may be further braided and/or connected between only two layers or all of the layers. Additionally, different connecting wires 116 may be connected to different pairs of stent layers.

It should also be appreciated that different materials may be utilized for the connecting wire 116 other than Nitinol which was previously discussed. As further examples, the connecting wire 116 may be composed of DFT or tantalum wire. It has been shown, however, that Nitinol or DFT connecting wires 116 may provide better stent diameter recovery to keep the layers 102, 104 together when compared with tantalum connecting wires 116.

The disclosure also includes a method of manufacturing a stent by pre-shaping or heat setting a shape to a connecting member 116 and then connecting and/or braiding/weaving the connecting member 116 to one or more stent layers.

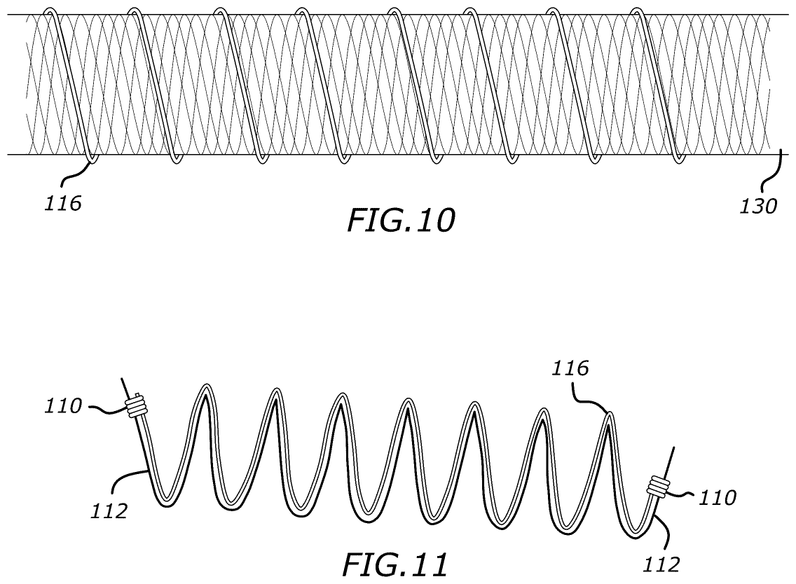

One specific example method is described with regard to the dual layer stent 100 of FIGS. 2A-7, though it is applicable to any of the example of this specification. In such a method, a shape memory connecting wire 116 (e.g., Nitinol) may be wrapped around a fixture or mandrel 130, as seen in FIG. 10. The connecting wire 116 may be wrapped to have a coil angle that matches that of one of the wire portions of the outer layer 102. The mandrel 130 may include guides, grooves, or similar physical features to help achieve a desired helical diameter and pitch. Optionally, the mandrel 130 may have a diameter that is larger than a mandrel that the remaining stent layers 102, 104 are braided on. The connecting wire 116 may then be heat set on the mandrel 130 to retain this coil shape and size. The connecting wire 116 can be further processed or finished as needed, such as via polishing, passivating, etching, or pickling.

The woven outer layer 102 and inner layer 104 can then be brought together so that the inner layer 104 is positioned and aligned within the outer layer 102, or can be optionally braided on top of each other. These layers 102 and 104 may be heat set on a mandrel to set a predetermined radial size in an expanded configuration. Optionally, this mandrel diameter size may be smaller than that of the mandrel 130. The connecting wire 116 can then be woven through both layers 102 and 104 to generally following a similar path adjacent to one of the outer wires 112, but otherwise passing over and under both wires 112 and 114. The result is that the connecting wire 116 may have its helical, heat-set form that is interwoven with and generally matches with that of one or both of the other two layers 102, 104 (illustrated in FIG. 11). Alternatively, the connecting wire may be braided in a helical direction that is rotationally opposite (opposing pitch) to that of the outer wires 112. Alternatively, the connecting wire 116 may be located between the layers 102 and 104 without interweaving into the layers.

When the connecting wire 116 is in its desired position, wire ties or coils 110 (or other previously discussed connection mechanism) can be formed on each end of the connecting wire 216, as seen in FIG. 11. The wire ties 110 can be formed by wrapping a wire (e.g., tantalum) around both the connecting wire 116 and a portion of the outer wire 112. Alternatively, the wire of the tie 110 can also be wrapped around the inner wire 114. Alternatively, the ends of the connecting wire 116 may be wrapped around the outer wire 112 to form the wire ties 110, however, a non-shape memory material may provide greater resistance to deformation and thereby provide a stronger connection point. Additionally, wire ties 110 (or similar connections) may be included at other locations along the length of the connecting wire 116.

It should be appreciated that the pitch of the connecting wire 116 may vary along the length of the stent 100 as the connecting wire 116 is woven through the layers 102, 104 of the stent 100. As an example, a pitch of a first winding of the connecting wire 116 may be different from a pitch of a second winding of the connecting wire 116. Additionally, the direction of the winding may vary in different examples, with one example using right hand winding and another example using left hand winding. Further, the OD of the winding of the connecting wire 116 may vary in different examples.

The stent 100 of FIGS. 2A-7 includes five relatively larger loops 106 and five relatively smaller loops 109. However, additional numbers of loops and sizes of loops are also possible. In that regard, any of the stents described in this specification may include a plurality of larger loops 106 and smaller loops 109 that form an alternating pattern on one or more of its ends. For example, FIG. 13 illustrates a stent 142 with four pairs of larger and smaller alternating loops 109. In another example seen in FIG. 14, a stent 144 may include six pairs of larger and smaller loops 106, 109. Additionally, examples with only one size of end loop are also possible (e.g., all loops may be of a substantially uniform size). For example, FIG. 15 illustrates a stent 146 that includes eight pairs of similarly size loops 106. The examples of FIGS. 13-15 may be particularly suited for stents having a diameter greater than 5.00 mm, such as between about 6.0 mm to 8.0 mm, to improve stent opening and stability.

The long flares/loops 106 and short flares/loops 109 can each be oriented at about a 60-degree angle (relative to a horizontal plane extending through the axial/radial middle of the stent). The flare/loop sizes can vary based on the size of the stent as well. In various examples, the stent is sized from about 2.5-5 mm in diameter. In some examples, the stent may be sized greater than 5 mm in diameter, such as between 6-8 mm in diameter. This particular size may fit neurovascular arteries, which are smaller than arteries in the majority of the vasculature, and provide benefit as a scaffolding stent used to provide support against a neck region of an aneurysm for subsequent devices (e.g., embolic coils, or other occlusive agents) used to fill the aneurysm. Proper apposition of the stent may be particularly helpful in this target therapeutic regimen to ensure the stent does not migrate away from the aneurysm site, which could then allow embolic material to migrate when left without a supporting scaffold.

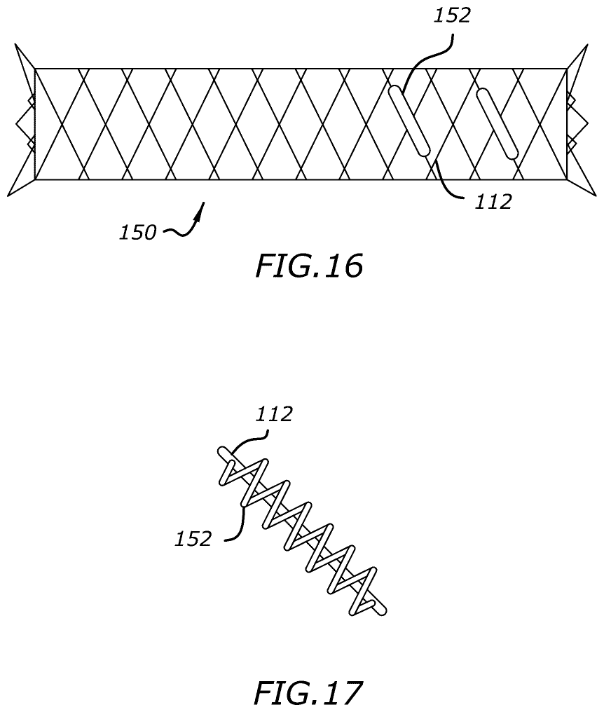

Any of the stent examples of this specification may include one or more reinforcing elements to help further increase the force with which a stent radially expands. For example, FIGS. 16 and 17 illustrate aspects of a stent 150 that is similar to stent 100 or 140. However, one or more regions of the stent 150 may include reinforcement elements 152 positioned over stent wire 112 (which may be a DFT wire 10) to introduce increased strength and stiffness along the one or more regions. It should be appreciated that the number, size, positioning, and orientation of any such reinforcement elements 152 may vary in different examples.

In typical braided stents, it can be difficult to fully expand the proximal end of the stent once the remaining portion of the stent is deployed. This can be particularly due to tortuous vasculature in which a stent is deployed. This problem may be magnified as stents are designed to be less stiff and more flexible, such as by using DFT wires 10 that are heat set. Therefore, introducing one or more reinforcement elements 152 along a portion of the stent 150, such as a proximal, distal, or medial region of the stent 150, may help augment opening force along this region, promoting ease of deployment. These reinforcement elements 152 can also be used in combination with the previously discussed connecting wire 116 so that both components provide radially expansive force on the stent 150.

The reinforcing element 152, in one example, may comprise a coil as is shown in greater detail in FIG. 17, where the reinforcing coil is wound around the DFT stent wire 112 of the stent 150. In other examples such as shown in FIG. 16, the reinforcing element 152 may comprise a tube that is placed over the DFT wire 112 along one or more regions of the stent. In one example, the reinforcing element 152 may be attached to the wire 112 (e.g., via adhesive or welding) to fix the location. In another example, the reinforcing element 154 may not be fixed and may be free to move (e.g., by sliding and/or rotating). In another example, the reinforcing element 154 may be another linear wire element which is attached to a portion of the DFT wire 112 to “thicken” the associated DFT wire segment.

The reinforcing element 152, in one example, may be made of a strong shape memory material. A preferred example is Nitinol (e.g., either a Nitinol coil or a Nitinol tube), but other examples can include cobalt-chromium or stainless steel.

Where the reinforcing element 152 is a coil, as is shown in FIG. 17, this coil may have an associated stiffness or k-value associated with it. This stiffness/k-value may depend on a number of attributes including the material composition, the thickness of the coil, and how close wound the reinforcing coil is (i.e., the pitch). A higher k-value could be effected, for instance, by utilizing a relatively stiff material (e.g. radiopaque material such as gold, platinum, tungsten, palladium, tantalum, or non-radiopaque metals that are stiff), by using a closely-wound pitch for the coil, and/or by adjusting properties of the coil (e.g., the thickness of the wire comprising the coil, the overall width of the coil, and the overall length of the coiled reinforcing element 152).

The portion of the wire 112 which is underneath the reinforcing element 152 may have its own associated stiffness of k-value, as the wire forming the reinforcing element 152 may have its own corresponding “springiness” due to being wound in a helical, longitudinal manner along the stent 150. Note, this “springiness” will increase as the stent 150 is compressed and may help to propel the stent 150 open upon deployment. The k-value of the wire 112 will depend on the associated stiffness of DFT wire, the diameter of the wire, and the pitch of the wire comprising the DFT stent 150 (in other words, the helical/longitudinal wind pattern used to mechanically wind the stent 150).

The stent region shown in FIG. 17, where the reinforcing coil sits over a portion of the wire 112, can be thought of as two parallel springs and Hooke's law would yield a corresponding stiffness. Where the wire 112 has an associated stiffness k₁ and the reinforcing coil 152 has an associated stiffness k₂, the overall stiffness of this region will then be (k₁+k₂), in other words the combined stiffness will be higher. In this way, the reinforcing element 152 may serve to increase the associated stiffness at that region. This increased stiffness has certain advantages, for instance strengthening a particular region of the stent 150 to augment deployment force (helping the stent open) and promoting apposition against the vessel wall along the reinforced section.

Another advantage is that the augmented stiffness and the enhanced area that the reinforcing element takes up across the underlying wire will help adjacent cells of the stent 150 open. If adjacent cells cannot sufficiently open, these cells will contact the reinforcing element 152 (which has a higher surface area than the underlying and surrounding wire 112), and this contact force can help these other cells open.

The reinforcing element 152 can be placed in one or more regions along the DFT stent 150. For instance, it can be placed in roughly equidistant intervals (or alternatively, in random locations) over the length of the stent 150 to promote a consistent expansion and consistent enhanced stiffness across the entirety of the stent. Alternatively, it can be placed along solely the proximal section of the stent 150 (as shown in FIG. 16), in one or more locations along the proximal section in order to enhance strength and opening in the proximal region of the stent.

In an example, a pair of reinforcing elements 154 may be positioned at a location along the radial circumference of the stent 150. In such an example, the reinforcing elements 152 may be aligned with each other along a longitudinal axis extending through the length of the stent 150. Additional reinforcing elements 152 may be positioned in such an example at various other radial locations around the circumference of the stent 100, such as on the opposing side, as necessary to augment the stiffness of the stent 150.

In other examples, the locations of the reinforcing elements 154 may vary from that shown in the figures. For example, in some examples, the reinforcing elements 152 may instead or additionally be positioned at or near the distal region of the stent 150. As a further example, the reinforcing elements 152 may instead be positioned on the opposing winds such that they are angled differently than is shown in the figures.

The reinforcing element 152 can be added in a variety of ways to the DFT wire 112 of the stent 150. The following techniques can be used regardless of whether the DFT stent 150 comprises solely one DFT wire, or a plurality of DFT wires. In one example, the reinforcing element 152 may be slid over the respective wire segment before or during the winding procedure used to wind the stent 150.

In another example, the wire can be cut near the region where the reinforcing element 152 is added to the wire, and once the reinforcing element 152 is appropriately placed, the wire may then be soldered or welded to the other cut section of the wire to reattach the two wire segments. One advantage of placing this wire attachment location near the reinforcing element 152 is that this will thicken the associated wire segment, which can help keep the reinforcing element 152 in a particular location and keep it from moving around.

In one example, the reinforcing element 152 is a Nitinol coil having an inner diameter of about 0.003 inches and an outer diameter of about 0.0065 inches. Where plural reinforcing elements 152 are used, they can be spaced in various ways, for instance one wire wind can separate two elements 152, more wire winds can separate the two elements, or the elements 152 can be spaced directly adjacent each other at adjacent winds.

While the example of the stent 100 discussed primarily with regard to FIGS. 2A-7, as well as other locations, primarily discusses the use of a DFT wire 10 to make up the outer layer 102, Alternatively a wire composed entirely of radiopaque material can be used instead, such as gold, platinum, tungsten, platinum-tungsten, palladium, iridium, platinum-iridium, rhodium, tantalum, barium sulfate, bismuth subcarbonate, bismuth oxychloride, bismuth trioxide or combinations thereof. Hence, one aspect of the disclosure includes a stent having a first braided layer braided from one or more radiopaque wires and a second braided layer braided from one or more shape memory wires, where the two layers are connected to each other.

In one example, the disclosure includes a stent comprising at least one layer braided from one or more wires; the at least one braided layer forming a stent body having a tubular shape and having a plurality of longer loops and a plurality of shorter loops disposed at its proximal end, its distal end, or both its proximal and distal ends; wherein the plurality of longer loops and the plurality of shorter loops form an overlapping and alternating pattern; and, a radiopaque marker positioned on at least one of the plurality of longer loops, adjacent to the stent body such that an adjacent shorter loop of the plurality of shorter loops does not contact or move across the radiopaque marker.

The term shape set is used within this specification to refer to an imparted secondary shape on a wire or similar component that is composed of a shape memory alloy such as Nitinol. Typically, such shape setting may occur by the application of heat when a component is placed in a desired shape that the component may return to after deformation within certain temperatures.

The present specification includes examples of different types of stents that comprise one or more connecting wires 116. For example, the stents may be a single layer, dual layer, or a plurality of layers, and each of the one or more layers may be braided from one or more wires for each. As discussed with regard to FIGS. 5 and 7, it may be desirable to connect or fix such a connecting wire 116 to the outer wire 112 of outer layer, the inner wire 114 of the inner layer 104, or the wires that make up any of the other layers, if present.

When possible, it may be desirable to create a coil with the end of the connecting wire around a stent wire (outer wire 112 may be used an example hereafter, but the inner wire 114 or any other layer's wire may also be used). This may be easily achievable if the connecting wire is composed of a relatively soft material, such as tantalum. However, harder materials, such as Nitinol, may be difficult to form such an end coil due to its stiffness in wire form. Hence, other connection approaches may improve ease of manufacture and reliability in use.

One connection approach is to apply an adhesive between connecting wire 116 and 112. For example, a UV curing adhesive may be used. Generally, such an adhesive may work well to bond the two wires 112, 116 together. However, in some circumstances, additional processing of the stent may be desirable, such as electropolishing and/or applying different coatings (e.g., a functionalized coating). Depending on the additional processing, the adhesive may soften and/or cause the connection between the two wires 112, 116 to weaken or break. In that respect, it may be desirable to further reinforce the adhesive connection or even replace the adhesive with other connection mechanisms.

The present disclosure includes connection mechanisms that include any combination of adhesive, coils, laser welding, beads, sleeves, tubes, or similar structures. For example, a first wire of a first material and a second wire of a second material may be connected or bound together via one or more of the following: a third wire or wires coiled around both the first and second wires, one or a plurality of sleeves positioned around the first and second wires, and/or one or a plurality of tubes positioned around the first and second wires. Adhesive may optionally be applied to the first and second wires at a region that the third wire(s), sleeve(s), or tube(s) are located. A stop member may optionally be included with any of the prior examples where the stop member is located on only the first wire adjacent to the third wire(s), sleeve(s), or tube(s), on only the second wire adjacent to the third wire(s), sleeve(s), or tube(s), or on both the first wire and the second wire adjacent to ends of the third wire(s), sleeve(s), or tube(s). Each of the stop members may include one or more wire coils, one or more laser weld locations, one or more beads, one or more sleeves, one or more tubes, or similar shapes. In any of the prior examples, the first material may be a shape memory alloy, such as Nitinol, and the second material may be any of the DFT wire examples of this disclosure. In any of the prior examples, the first wire may be about parallel with the second wire, both extending in generally the same orientation and direction (e.g., such as connecting wire 116 with outer wire 112 in FIG. 2).

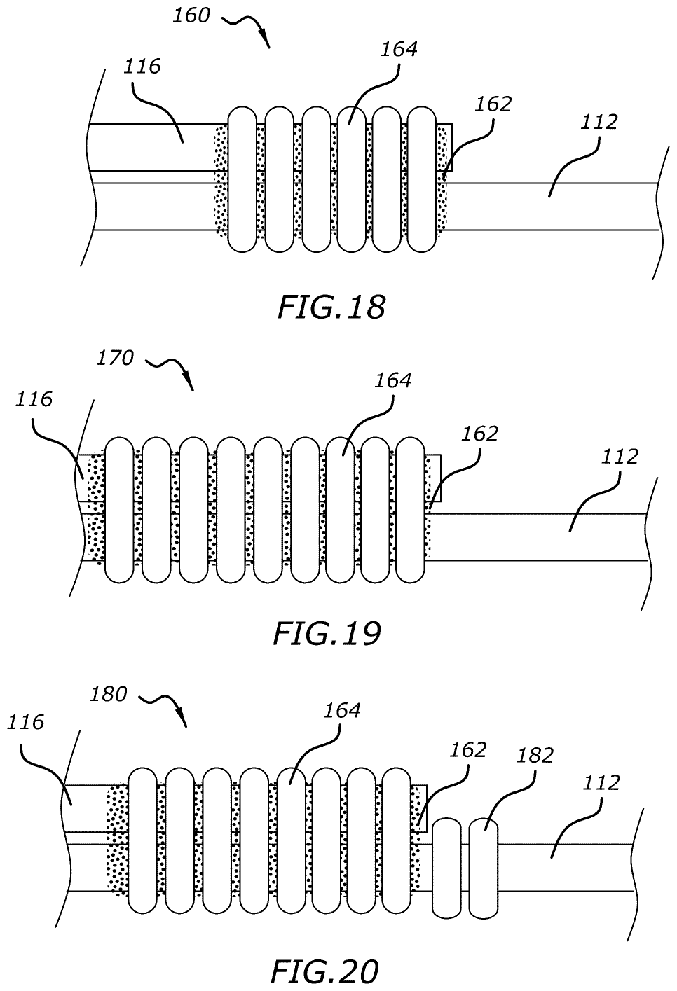

FIGS. 18-24 illustrate several different specific example connection mechanisms that may connect any two stent wires together, such as the previously discussed connecting wire 116 to an outer wire 112 (or other wire of another layer) of a stent. In the present examples, the connecting wire 116 is composed of Nitinol and the outer wire 112 is a DFT wire as discussed earlier in this specification, however, other materials may also be used for each wire 112, 116. Additionally, if an adhesive 162 is used, these examples may also further reinforce an adhesive 162 that connects both wires 112, 116, but the structures may also be used without the adhesive.

The example of FIG. 18 illustrates a wire connection structure 160 comprising a primary wire coil 164 placed around adhesive 162, the end region of the connecting wire 116, and the outer wire 112. The primary wire coil 164 may be composed of a flexible metal, such as tantalum, that is wrapped around wires 112, 116 several times. In the present example, six loops are formed, but any number of loops can be formed, such as 1, 2, 3, 4, 5, 6, 7, 8, 9, 10 or more. The example wire connection structure 170 of FIG. 19 illustrates a specific example of nine loops formed. Note that for simplicity, the ends of the wire used to form the primary wire coil 164 are not shown.

Alternatively, the primary wire coil 164 may be a sleeve, tube or similar structure that is crimped or otherwise attached to the wires 112, 116 for any of the examples discussed in FIGS. 18-25B. Alternatively, the adhesive 162 may be omitted for any of the examples discussed in FIGS. 18-25B. The end region of connecting wire 116 may include any portion of length between 0 mm and 5 mm, or 10% of length, from a free end of the connecting wire 116 for any of the examples discussed in FIGS. 18-25B. However, any connection location along connecting wire 116 (or any alternative wires) may also be possible for any of the examples discussed in FIGS. 18-25B.

Depending on what additional processing is performed on the stent during its manufacturing process, the adhesive 162 may weaken enough that the connecting wire 116 may move longitudinally back and forth within the primary wire coil 164, particularly when the stent expands or contracts. Alternatively, if no adhesive 162 is used, the connecting wire 116 may also move longitudinally relative to the outer wire 112. Hence, it may also be desirable to include further structures, also referred to as “stop members” in this disclosure, near either or both ends of the primary wire coil 164 to help prevent this longitudinal movement.

For example, FIG. 20 illustrates a wire connection structure 180 with a stop member in the form of a primary wire coil 164 having a plurality of loops (e.g., 8) over adhesive 162 and wires 112, 116, as similarly discussed. A secondary coil 182 is placed around only the outer wire 112 adjacent to the primary wire coil 164 and adjacent to the end of the wire 116. Therefore, if the outer wire 112 moves relative to the primary wire coil 164, at least in one direction, it will abut the secondary coil 182 and be prevented from moving further. Again, the free ends of the secondary coil 182 are not shown for simplicity. Depending on the size of the wire of the secondary coil 182, it may form a diameter large enough to also block or abut the end of the connecting wire 116, thereby also preventing it from moving, at least in one direction, relative to the outer wire 112. Note, adjacent in this context means next to and either in contact with or within 5 mm of each other.

The secondary coil 182 may comprise a wire composed of bendable material (e.g., a metal such as tantalum). Alternatively, the coil 182 may instead be a tube, sleeve, a laser weld, a bead, an enlargement, or similar unitary shape fixed to the wire 112.

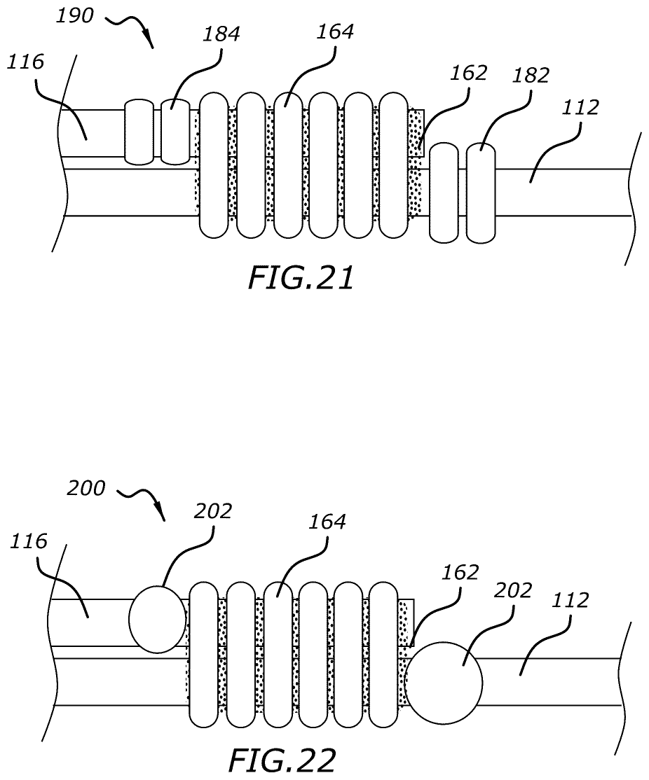

The secondary coil is not limited to only the location of FIG. 20. It may be positioned on either side of the primary wire coil 164 and on either wire 112, 116. For example, FIG. 21 illustrates a wire connection structure 190 having a first secondary coil 182 on the outer wire 112, adjacent to the end of the connecting wire 112, and a second secondary coil 184 on the opposite side of the first secondary coil 182 and around the connecting wire 116. An additional wire may be fixed on the outer wire 112 on the opposite side of the coil 164. A further additional secondary coil may be fixed at the end of the connecting wire 116. In other words, there are four possible locations where a secondary coil can be located, and such coils may be included in any combination of locations.

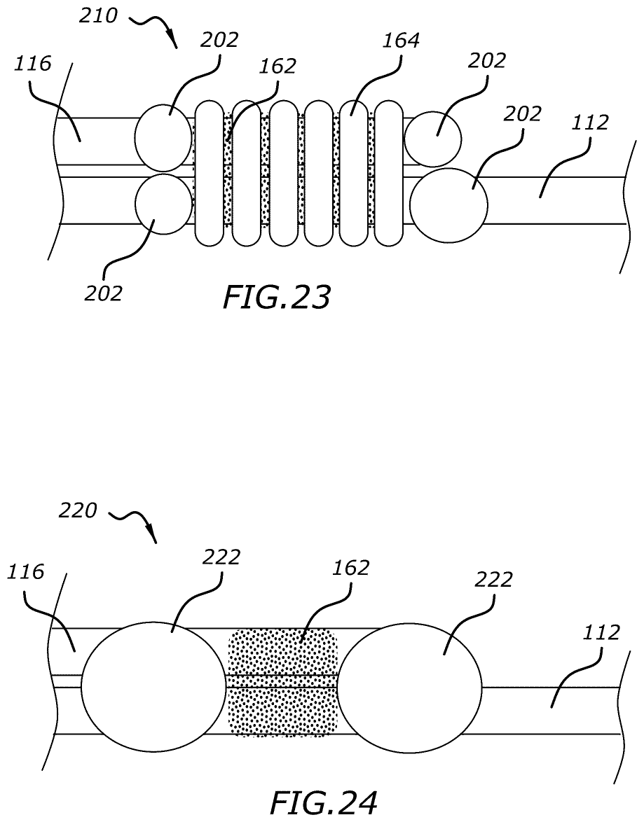

While a coil or crimped sleeve/tube may be used as described above for the secondary coil 182, 184, an enlargement of any kind may also be used. For example, a bead 202 may be fixed to one or more of the wires 112, 116 in similar locations adjacent to the primary coil 164 as previously discussed. In one example, the bead 202 may be formed from welded material at any of the locations specified for the secondary coil above. For example, the wire connection structure 200 of FIG. 22 illustrates a first welded bead 202 on the outer wire 112 and a second welded bead 202 on the connecting wire 116. In another example, FIG. 23 illustrates four welded beads on each side of the primary wire coil 164 and on each wire 112, 116, though again, any combination of locations may be included.

As seen in FIG. 24, a wire connection structure 220 may not include the primary wire coil 164 and may instead include one or more weld locations 222. The present example includes two weld locations 222 that encompass the two wires 112, 116 located near or at the end of the connecting wire 116 and adjacent to the first weld location 222. In this example, a gap or longitudinal space is created between the two weld locations 222 in which the adhesive 162 is located. While two weld locations 222 are shown, the wire connection structure 220 may include 1, 2, 3, 4, 5, or more weld locations 222. These weld locations 222 may be immediately next to each other or spaced apart from each other. In one example, some weld locations may be immediately adjacent to each other and others form a longitudinal space or gap relative to each other (e.g., two weld locations 222 immediately adjacent to each other followed by another weld location 222 spaced apart from the first two weld locations 222). Optionally the adhesive 162 may be included between the weld locations 222 and/or on either side of a weld location 222 (i.e., not necessarily only between two weld locations 222).

It should be understood that the examples of FIGS. 18-24 may be included at only one end of the connecting wire 116 or at both ends of the connecting wire 116. Hence, one end of the wire may have an opposite orientation as what is shown in those figures. Additionally, the wire connection structures were discussed between an outer stent wire and a connecting wire, these techniques can be used for connecting any two wires of a stent of any number of layers together.

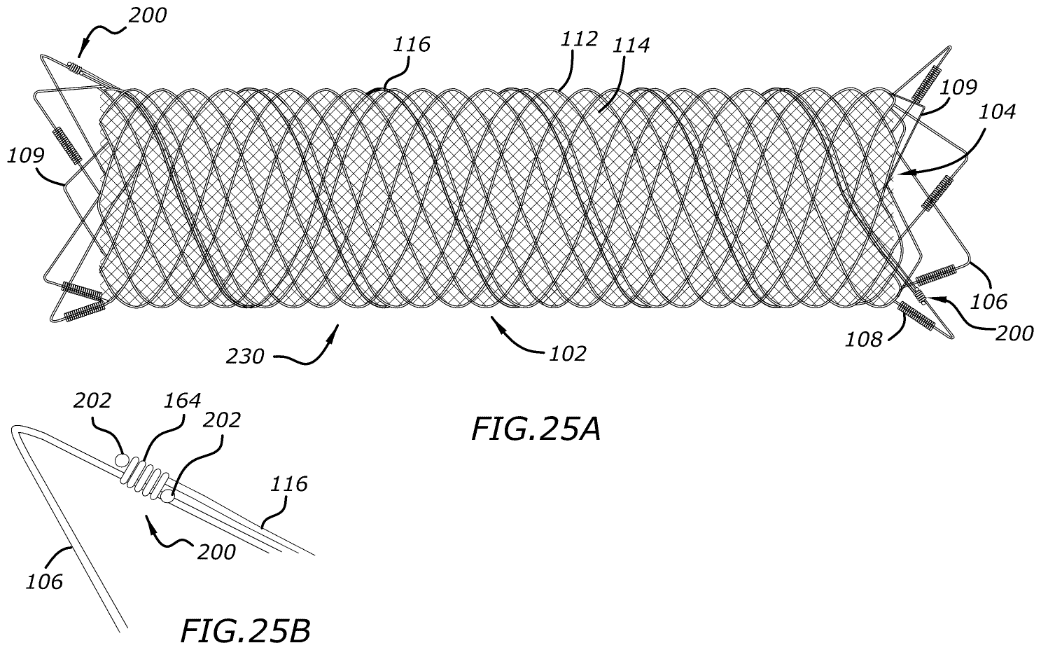

While the previously described examples of wire connection structures may be positioned along the length of the tubular body portion of a stent, such as the locations noted with regard to the stent 100 in FIGS. 2A, 5, and 7, other locations are also possible. For example, FIG. 25A illustrates a stent 230 that is generally similar to the previously described stent 100 except that the connecting wire 116 is connected to an end loop 106 of the stent 230 via the connection structure 200. FIG. 25B illustrates a magnified view of one of the loops 106 to which the connection structure 200 connects the connecting wire 116.

While FIG. 25A illustrates both ends of the connecting wire 116 connected to a loop 106, the connecting wire 116 may alternatively be connected at one end to a loop 106 and at the other end to a location along the body (e.g., outer wire 112) similar to that shown in FIG. 2. While the connection structure 200 is shown, any connection structure of the present disclosure may alternatively be used. The connection structure 200 may be connected at any position on the loop, such as the base of the loop 106, a middle location of the loop 106, or near/at a tip of the loop 106.

Locating the connection structure 200 on or at the loop 106 may have several benefits, depending on the stent design. For example, in the present FIG. 25A, 25B example, the stent has a tubular shaped inner layer 104 and a tubular shaped outer layer that may be in close radial proximity to each other. Connection structures located along the main tubular portion may act as a point of friction between the inner layer 104 and the loops 106 of the outer layer, particularly when expanding or contracting the stent. However, locating a connection structure 200 at the loops 106 of the stent may space the connection structures 200 away from the inner layer 104 and thereby reduce some friction between the inner layer 104 and outer layer 102. Additionally, such a connection structure 200 may eliminate the need for a separate radiopaque coil to be placed on a loop 106, and thereby simplify the manufacturing process.

It should be understood that different aspects of the examples of this specification can be interchanged and combined with each other. In other words, additional examples are also specifically contemplated by combining different features from different examples. Therefore, while specific examples are shown in the Figures, it is not intended that the invention necessarily be solely limited to those specific combinations.

What is claimed is:

1. A stent comprising:
a first wire;
a second wire; and,
a wire connection structure fixed at an end region of the second wire and fixed to the first wire.

2. The stent of claim 1, wherein the first wire is about parallel to the second wire.

3. The stent of claim 1, wherein the first wire is braided to form a first stent layer and wherein the second wire is a connecting wire interwoven with the first wire.

4. The stent of claim 3, wherein the connecting wire is interwoven with the first stent layer and with a second stent layer.

5. The stent of claim 3, wherein the first wire comprises a DFT wire and wherein the second wire comprises a Nitinol wire.

6. The stent of claim 1, wherein the wire connection structure comprises a primary wire coil wrapped around the first wire and the second wire, near the end region of the second wire.

7. The stent of claim 6, wherein the primary wire coil comprises 1, 2, 3, 4, 5, 6, 7, 8, 9, or 10 loops.

8. The stent of claim 7, wherein the primary wire coil is composed of a tantalum wire.

9. The stent of claim 7, further comprising adhesive located underneath the primary wire coil.

10. The stent of claim 7, further comprising one or a plurality of stop members connected to the first wire, the second wire, or separately to the first wire around the second wire; wherein the one or plurality of stop members are adjacent to the primary wire coil.

11. The stent of claim 10, wherein the first wire comprises a first stop member adjacent a first end of the primary wire coil and a second stop member adjacent a second end of the primary wire coil; and wherein the second wire comprises a third stop member adjacent the first end of the primary wire coil and a fourth stop member adjacent the second end of the primary wire coil.

12. The stent of claim 10, wherein the one or plurality of secondary stop members comprise one or a plurality of secondary coils, one or a plurality of sleeves, one or a plurality of tubes, one or a plurality of beads, one or a plurality of welds, or one or a plurality of attached enlargements.

13. The stent of claim 10, wherein the wire connection structure comprises a first weld that is welded to the first wire and the second wire, and a second weld wired to the first wire and the second wire at the end region of the second wire.

14. The stent of claim 13, wherein the first weld is spaced apart from the second weld;
and wherein adhesive is located on the first wire and the second wire between the first weld and the second weld.

15. The stent of claim 4, wherein the connecting wire is connected to an end loop of the first stent layer formed from the first wire.

16. The stent of claim 15, wherein the wire connection structure further comprises a first wire connection structure connecting a first end region of the connecting wire to a first end loop of the stent, and a second wire connection structure connecting a second end region of the connecting wire to a second end loop of the stent.

17. A stent comprising:
a first wire comprising a first material;
a second wire that is about parallel to the first wire and comprising a second material; and,
a wire connection structure fixed at an end region of the second wire and fixed to the first wire; the wire connection structure comprising a primary wire coil and one or more stop members located longitudinally adjacent the primary wire coil.

18. The stent of claim 17, wherein the stop members comprise one or a plurality of secondary coils, one or a plurality of sleeves, one or a plurality of tubes, one or a plurality of beads, one or a plurality of welds, or one or a plurality of attached enlargements.

19. The stent of claim 18, further comprising adhesive located at least underneath the primary wire coil.

20. A stent comprising:
a first wire;
a second wire; and,
a wire connection structure means for fixing an end region of the second wire to the first wire.
